(19) United States
(12) Patent Application Publication
(10) Pub. No.: US 2024/0285709 A1
(43) Pub. Date: Aug. 29, 2024

(54) MONK FRUIT EXTRACT AND METHOD FOR PRODUCING SAME

(71) Applicants: FONTEC R&D CO., LTD., Okayama (JP); DAICEL CORPORATION, Osaka (JP)

(72) Inventors: Takahito ICHI, Maniwa-shi Okayama (JP); Akinobu MATSUYAMA, Tokyo (JP); Lirui PAN, Tokyo (JP)

(21) Appl. No.: 18/569,614

(22) PCT Filed: Mar. 23, 2022
(86) PCT No.: PCT/JP2022/013627

§ 371 Date: Dec. 13, 2023

(30) Foreign Application Priority Data
Jun. 14, 2021 (JP) 2021-098464

(51) Int. Cl. A61K 36/42; A23L 27/30; A61K 47/28

(52) U.S. Cl. CPC A61K 36/42; A23L 27/36; A61K 47/28

(57) ABSTRACT

To provide a monk fruit extract that has the high content of mogroside V, has good sweetness quality similar to sucrose, and has a less undesirable flavor such as bitterness and that can be easily and economically obtained on a large scale.

A monk fruit extract of the present disclosure includes (a) 65% by mass to 85% by mass of mogroside V in the extract, (b) 1% by mass or less of a protein in the extract, and (c) 0.1% by mass or less of a polyphenol in the extract.

### TECHNICAL FIELD

The present invention relates to a monk fruit extract and a method for producing the same.

### BACKGROUND ART

Monk fruit (Lou Han Guo, scientific name; Siraitia grosvenorii (Swingle) C.) is a climbing perennial plant that belongs to the Cucurbitaceae family. Monk fruit reaches up to 5 m in length and has tubers below the soil. Monk fruit is distributed in southern China and preferably grows on soft, humus-rich mountain slopes or the like in cool, foggy regions with large temperature differences between day and night. Monk fruit is exclusively cultivated mainly in Guangxi in the region of Guilin in China. Monk fruit is effective for bronchitis, tonsillitis, pharyngitis, cough suppression, acute gastritis, constipation, and the like, and has been traditionally used as a folk medicinal fruit.

In recent years, it becomes evident that monk fruit has pharmacological effects (functionality) such as an antioxidant effect (anti-aging effect, anti-cancer effect) which eliminates active oxygen (free radical), and a blood sugar-lowering effect, thus being effective in preventing and treating hypertension, diabetes, and the like.

Currently, as monk fruit compositions, there are generally three types of products on the market, namely, “low content type”, “heat-treated type”, and “refined monk fruit”. Various patent applications have been filed for these products. A monk fruit product of the “low content type” is a monk fruit preparation prepared by adding approximately 0.8% by mass of a sugar alcohol such as erythritol to a composition including approximately 33% by mass of a mogroside group compound. In this case, this product includes only about 0.264% by mass of the mogroside group compound, and it is estimated that the concentration of the mogroside group compound in a final food product, to which the monk fruit preparation is added, is about 5 to 20 ppm. Thus, it is considered difficult for consumers to expect an improvement in sweetness quality and functionality caused by the monk fruit.

In a monk fruit product of the “heat-treated type”, components such as proteins are thermally denatured by heating, resulting in a brownish color, which leads to the speculation that the sweetness quality and functionality are significantly deteriorated.

A monk fruit product of the “refined monk fruit” is mainly distributed in the market as a food additive. However, current industrial production methods can only purify the mogroside group compound to the content of about 5% by mass to 60% by mass. Thus, when the product is added to food, it causes a strange taste similar to that of Chinese herbal medicine.

At present, there is a strong demand from customers and consumers for the development of a highly refined monk fruit sweetener composition that improves these problems. Thus, in order to meet such demands, various techniques for improving monk fruit products have been reported. For example, Patent literature 1 discloses a monk fruit (Siraitia grosvenorii) extract which includes: (1) 0.01% by mass to 70% by mass of mogroside V; and (2) 5% by mass or less of a water-insoluble component, with a ratio of the “insoluble polyphenol content/total polyphenol content” of 1/2 or less.

Further, Patent literature 2 discloses a composition including a monk fruit glycoside represented by a predetermined general formula. The monk fruit glycoside has a sweet taste and an antioxidant effect.

Further, Patent literature 3 discloses an extract derived from a raw fruit of the monk fruit obtained by treating raw fruit juice or a liquid extract of the monk fruit with one or more enzymes selected from a pectinase enzyme, a cellulase enzyme, a hemicellulase enzyme, a xylanase enzyme, a protease enzyme, and an amylase enzyme. The turbidity of the resulting aqueous solution caused by a fruit-derived solid is defined as the absorbance at a wavelength of 660 nm (10 mm quartz cell) in the shaded area in FIG. 1 of the provided drawings. As a result, the monk fruit extract exhibits a clear light color and has a good flavor.

Further, Patent literature 4 discloses a method for producing a monk fruit powder including: an inclusion treatment step in which a mixing and stirring treatment is applied to an aqueous phase in which a monk fruit extract is present in a ratio of 0.2 to 2.0 parts by weight relative to 1 part by weight of a predetermined cyclodextrin; and a drying step in which the water content is removed from the aqueous phase subjected to the mixing and stirring treatment by rapid drying to form a powder of the cyclodextrin and the monk fruit extract.

In response to the market need for the development of a highly refined monk fruit sweetener composition, the present inventors have developed a method for producing a high-content monk fruit composition using an immobilized enzyme membrane method (Patent literature 5). This production method can achieve the mogroside V content of 65% by mass, and a product obtained by this production method has improved the sweetness quality compared to a product with the mogroside V content of 50 to 60% by mass.

### CITATION LIST

### Patent Literature

Patent Literature 1: Japanese Patent No. 6812032
Patent Literature 2: JP 2001-322934 A
Patent Literature 3: Japanese Patent No. 3547553
Patent Literature 4: JP H9-173006 A
Patent Literature 5: Japanese Patent No. 6612255

### SUMMARY OF INVENTION

### Technical Problem

In recent years, the need for naturally derived sweeteners for replacing sugar has increased, and there is a demand for a monk fruit extract which has the sweetness quality more similar to sugar with little change over time. Thus, the present inventors have studied on developing a monk fruit extract with the high content of mogroside V, in which components other than mogrosides, which cause deterioration in the sweetness quality, have been removed as much as possible. In this case, if the sole purpose is to increase the content of mogroside V, it can be achieved by repeating the purification process. However, the amount and compositional balance of components such as mogroside V, which play a useful role in causing the sweetness quality of monk fruit, are significantly disrupted, resulting in a risk of losing the good sweetness quality. Thus, it is necessary to consider the above-mentioned point while developing the monk fruit extract.

An object of the present disclosure is to provide a monk fruit extract with a high content of mogroside V, good sweetness quality similar to sucrose, and a less undesirable flavor such as bitterness. Further, another object of the present disclosure is to provide a method for producing the monk fruit extract.

### Solution to Problem

As a result of intensive studies to achieve the object of the present disclosure, the present inventors have found that the object of the present disclosure can be achieved by adjusting the amounts of mogroside V, a protein, and a polyphenol to the predetermined contents as described below.

That is, the present invention is as follows.
[1] A monk fruit (Siraitia grosvenorii) extract including:
   (a) 65% by mass to 85% by mass of mogroside V in the extract;
   (b) 1% by mass or less of a protein in the extract; and
   (c) 0.1% by mass or less of a polyphenol in the extract.
[2] The monk fruit extract described in the above [1], including:
   (a) 71% by mass to 85% by mass of the mogroside V in the extract;
   (b) 0.5% by mass or less of the protein in the extract; and
   (c) 0.08% by mass or less of the polyphenol in the extract.
[3] The monk fruit extract described in the above [1], in which the monk fruit extract has a total content of isomogroside V, mogroside IV, and siamenoside I of 7% by mass to 15% by mass.
[4] The monk fruit extract described in the above [1], in which the monk fruit extract includes 1% by mass or less of the protein, 0.1% by mass or less of the polyphenol, and 71% by mass to 85% by mass of the mogroside V, and a total content of the isomogroside V, the mogroside IV, and the siamenoside I of 5% by mass to 15% by mass.
[5] The monk fruit extract described in any of the above [1] to [4], in which the absorbance at a wavelength of 660 nm in a 10% by mass aqueous solution is 0.1 or less.
[6] A sweetener composition including the monk fruit extract described in any of the above [1] to [5] as an active ingredient.
[7] An antioxidant including the monk fruit extract described in any of the above [1] to [5] as an active ingredient.
[8] A food or drink product including the monk fruit extract described in any of the above [1] to [5] as an active ingredient.
[9] A quasi-drug including the monk fruit extract described in any of the above [1] to [5] as an active ingredient.
[10] A cosmetic including the monk fruit extract described in any of the above [1] to [5] as an active ingredient.
[11] A method for producing the monk fruit extract described in the above [1], including:
   a step of extracting monk fruit with an aqueous solvent;
   a step of applying a styrene-divinylbenzene-based synthetic adsorbent treatment method to an aqueous solvent liquid extract of the monk fruit obtained in the extraction step;
   a step of applying a weakly basic anion exchange resin treatment method to an adsorbent treated liquid obtained by application of the styrene-divinylbenzene-based synthetic adsorbent treatment method; and
   a step of applying a simulated moving bed method or a nanofiltration membrane method, or both thereof, to a solution including a monk fruit crude extract obtained by application of the weakly basic anion exchange resin treatment method, thereby adjusting a content of mogroside V to 65% by mass to 85% by mass.
[12] A method for producing a monk fruit extract, including:
   a step of extracting monk fruit with an aqueous solvent; and
   a step of applying one or more types of separation and purification resin treatment methods allowed to be used for food to an aqueous solvent liquid extract of the monk fruit obtained in the extraction step, in which
   the monk fruit extract includes:
      (a) 65% by mass to 85% by mass of mogroside V in the extract;
      (b) 1% by mass or less of a protein in the extract; and
      (c) 0.1% by mass or less of a polyphenol in the extract.
[13] The method for producing the monk fruit extract described in the above [12], including:
   a step of extracting the monk fruit with the aqueous solvent; and
   a step of applying a nanofiltration membrane method to a solution including a monk fruit crude extract obtained by applying the one or more types of separation and purification resin treatment methods allowed to be used for food to the aqueous solvent liquid extract of the monk fruit obtained in the extraction step.
[14] The method for producing the monk fruit extract described in the above [12] or [13], in which the one or more types of separation and purification resin treatment methods include a simulated moving bed method.
[15] The method for producing the monk fruit extract described in any of the above [12] to [14], in which a resin adsorbent used in the separation and purification resin treatment method is at least one selected from the group consisting of a synthetic adsorbent, an ion exchange resin, an ODS resin, and a Sephadex resin.

### Advantageous Effects of Invention

The monk fruit extract of the present disclosure has the high content of mogroside V, has the good sweetness quality similar to sucrose, and has the excellent taste quality with a less undesirable flavor such as bitterness. Further, the method for producing the monk fruit extract of the present disclosure makes it possible to obtain easily and economically, at the actual production level (industrial scale), a large amount of the monk fruit extract which has the high content of mogroside V, has the good sweetness quality similar to sucrose, and has a less undesirable flavor such off-flavor as aftertaste or bitterness.

### BRIEF DESCRIPTION OF DRAWINGS

FIG. 1 is a diagram illustrating a result of output intensity measurements of mogroside V and protein contents in the first column outlet first eluate in Example 1.

### DESCRIPTION OF EMBODIMENTS

The present disclosure will be described in detail below.

As a result of repeated trial and error, the present inventors have found for the first time that, in order to obtain a monk fruit extract that has good sweetness quality similar to sucrose and has a less undesirable flavor such as off-flavor as aftertaste or bitterness, it is not always good to have the higher content of mogroside V, which is the main component of monk fruit sweet taste, but rather it is important to set the content of mogroside V within a predetermined range and to keep the contents of a protein and a polyphenol included in the monk fruit extract low, thereby completing the present invention.

In the present disclosure, the monk fruit extract refers to an extract obtained by adding a solvent to monk fruit (Lou Han Guo, scientific name: Siraitia grosvenorii (Swingle) C.) and purifying the resulting extract. A form of the monk fruit extract is not particularly limited, and may include a liquid form, a powder form, a massive form, a semi-solid form, and the like.

Mogroside V (molecular formula: C₆₀H₁₀₂O₂₉) is the main component of a triterpenoid glycoside present in the monk fruit extract as a sweetener, and is represented by the following chemical formula.

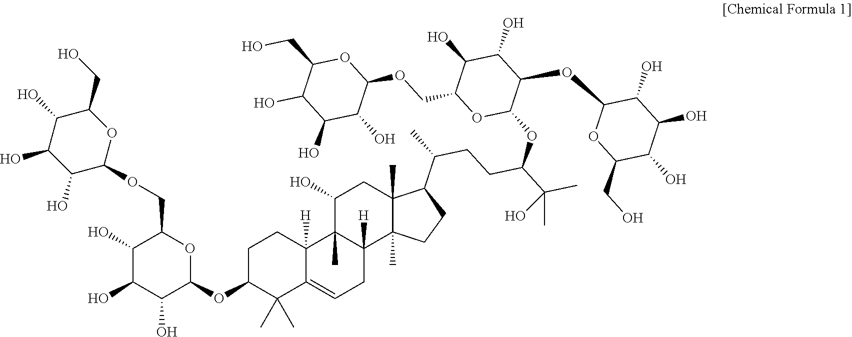

Isomogroside V (molecular formula: C₆₀H₁₀₂O₂₉), mogroside IV (molecular formula: C₅₄H₉₂O₂₄), and siamenoside I (molecular formula: C₅₄H₉₂O₂₄) are all known triterpenoid glycosides present in the monk fruit extract as sweeteners.

In the monk fruit extract of the present disclosure, the content of mogroside V is 65% by mass to 85% by mass, preferably 68% by mass to 85% by mass, more preferably 71% by mass to 85% by mass, relative to the total amount of the extract. If the content is less than 65% by mass, off-flavor and bitterness derived from a component other than mogroside V, a protein, and a polyphenol tend to increase, resulting in a decrease in sweetness quality compared to when the content is 65% by mass or more. On the other hand, if the content exceeds 85% by mass, the content ratio of isomogroside V, mogroside IV, and siamenoside I other than mogroside V, which have a sweet taste, decreases, resulting in a decrease in sweetness intensity. This also reduces the sweetness quality of the monk fruit extract.

The monk fruit extract of the present disclosure preferably includes a mogroside other than mogroside V in terms of improving the sweetness quality. Specifically, since isomogroside V, mogroside IV, and siamenoside I have higher off-flavor and bitterness than mogroside V, the total content of these components is preferably 7% by mass to 15% by mass, more preferably 7% by mass to 12% by mass, still more preferably 7% by mass to 10% by mass, relative to the total amount of the extract. If the content is less than 7% by mass, the sweetness intensity becomes low, making it difficult to obtain the sweetness quality similar to sucrose, and if the content exceeds 15% by mass, off-flavor and bitterness tend to increase, resulting in a decrease in the sweetness quality.

The contents of mogroside V and other mogrosides mentioned above can be measured as follows. First, approximately 0.05 g of a monk fruit sample is accurately weighed using a chemical balance, and the sample is added with distilled water up to 100 mL using a volumetric flask to obtain a solution used as an HPLC analysis sample. In the HPLC analysis [analysis conditions: column packing material; SHODEX Asahipak NH2P-50 4E (4.6 mm ID×250 mm), developing solvent; 75% CH₃CN/25% H₂O, isocratic mode, column temperature; 40° C., flow rate; 0.8 mL/min, detection wavelength; 203 nm, charge volume; 10 μL], calibration curves at a concentration of 100 ppm to 600 ppm are created for a standard of mogroside V and a standard of each of other mogrosides mentioned above to measure the contents of mogroside V and other mogrosides mentioned above in the sample.

In the monk fruit extract of the present disclosure, the protein content is 1% by mass or less, preferably 0.8% by mass or less, more preferably 0.5% by mass or less, relative to the total amount of the extract. If the content exceeds 1% by mass, off-flavor and bitterness tend to increase, resulting in a decrease in the sweetness quality. The protein content can be determined by the Kjeldahl method or a colorimetric method (BCA method, Bradford method, etc.). Since an increase in the amount of protein leads to a decrease in the sweetness quality of the monk fruit extract of the present disclosure, the lower limit of the content is 0% by mass.

In the monk fruit extract of the present disclosure, the polyphenol content is 0.1% by mass or less, preferably 0.09% by mass or less, more preferably 0.08% by mass or less, relative to the total amount of the extract. If the content exceeds 0.1% by mass, the brown color of the solution becomes darker, and off-flavor and bitterness derived from components other than mogroside V tend to increase, resulting in a decrease in the sweetness quality. Note that the polyphenol is a general term for a compound that has two or more hydroxyl groups bonded to an aromatic ring. Based on the chemical structure, the polyphenols are broadly classified into phenol carboxylic acids, phenol amines, anthocyanins, flavonoids, tannins, and the like. In the monk fruit extract, the polyphenol is involved in not only a taste (astringency, bitterness), a color, and aroma, but also health functionality such as an antioxidant effect. Since the polyphenol affects the color and sweetness quality of the monk fruit extract of the present disclosure, the lower limit of the content is 0% by mass.

The polyphenol content can be determined with the Folin-Ciocalteu method using a gallic acid standard solution according to “Determination of substances characteristic of green and black tea—Part 1: Content of total polyphenols in tea—Colorimetric method using Folin-Ciocalteu reagent” (ISO 14502-1:200).

The monk fruit extract of the present disclosure can include a component other than those described above as long as the effects of the present disclosure are not impaired. Examples of such a component include a mogroside other than mogroside V, a vitamin such as a B-group vitamin or vitamin E, various minerals such as iron, zinc, potassium, calcium, and magnesium, an amino acid, an oligopeptide, and water.

The monk fruit extract of the present disclosure has an absorbance at a wavelength of 660 nm in a 10% by mass aqueous solution of preferably 0.1 or less, more preferably 0.08 or less, still more preferably 0.05 or less. The extract having an absorbance of 0.1 or less can be made into a clear aqueous solution without turbidity. The absorbance can be determined by absorptiometry.

The monk fruit extract of the present disclosure can be used as an active ingredient of a sweetener that has the good sweetness quality similar to sucrose, and can impart or enhance sweetness to various products.

The monk fruit extract of the present disclosure is expected to have the high sweetness quality, functionality, physiological activity, and the like, and can be used in any product that requires the sweetness and/or functionality, such as an antioxidant, a food or drink product (including a food additive, a functional food, and a dietary supplement), a pharmaceutical drug, a quasi-drug, and a cosmetic.

Next, a method for producing the monk fruit extract of the present disclosure will be described.

The production method of the present disclosure includes:
a step of extracting monk fruit with an aqueous solvent;
a step of applying a styrene-divinylbenzene-based synthetic adsorbent treatment method to an aqueous solvent liquid extract of the monk fruit obtained in the extraction step;
a step of applying a weakly basic anion exchange resin treatment method to an adsorbent treated liquid obtained by application of the styrene-divinylbenzene-based synthetic adsorbent treatment method; and
a step of applying a simulated moving bed method or a nanofiltration membrane method, or both thereof, to a solution including a monk fruit crude extract obtained by application of the weakly basic anion exchange resin treatment method, thereby adjusting the mogroside V content to 65% by mass to 85% by mass.

Further, the production method of the present disclosure includes:
a step of extracting the monk fruit with the aqueous solvent; and
a step of applying one or more types of separation and purification resin treatment methods allowed to be used for food to the aqueous solvent liquid extract of the monk fruit obtained in the extraction step. Further, this production method can include a step of applying a nanofiltration membrane method to the solution including the monk fruit crude extract obtained by application of the one or more types of separation and purification resin treatment methods allowed to be used for food. The one or more types of separation and purification resin treatment methods may include a simulated moving bed method described below.

To carry out the production method of the present disclosure, it is first necessary to prepare a liquid extract of the monk fruit. To obtain this monk fruit liquid extract, the monk fruit may be extracted with an aqueous solvent. Specifically, in one embodiment, a fruit of the monk fruit and the aqueous solvent are placed in a container and heated at about 80° C. to 100° C., and then the residue is removed, followed by filtration, to prepare the liquid extract. Examples of the aqueous solvent include water, hydrous ethanol, and hydrous methanol. The sweetness quality of the monk fruit varies greatly depending on the harvest time (degree of ripeness), and a fully ripened fruit of the monk fruit is preferably used as it has good sweetness quality. Further, the fruit of the monk fruit may or may not be subjected to a drying treatment. However, the fruit is preferably not dried.

Next, a separation and purification resin treatment method allowed to be used for food is applied to the aqueous solvent liquid extract of the monk fruit obtained in the extraction step. Applying the separation and purification resin treatment method can effectively remove, in particular, highly polar (highly water-soluble) peptides, proteins, polyphenols, and the like, thereby purifying the liquid extract. A separation and purification resin allowed to be used for food is not particularly limited, and typical examples thereof include a styrene-divinylbenzene-based synthetic adsorbent. The styrene-divinylbenzene-based synthetic adsorbent is a synthetic adsorbent having a skeleton of a styrene-divinylbenzene copolymer or a modified product thereof. The pore radius of the styrene-divinylbenzene synthetic adsorbent is 50 Å to 400 Å, preferably 70 Å to 300 Å, and more preferably 90 Å to 260 Å.

The resin adsorbent used in the separation and purification resin treatment method can be at least one selected from the group consisting of a synthetic adsorbent, a cation exchange resin, a ODS resin, and a Sephadex resin. If the base resin structure of the resin adsorbent is styrene-based, examples of such an adsorbent include a synthetic adsorbent of DIAION HP series (e.g., HP20, HP20SS, HP21, etc.; all trade names, manufactured by Mitsubishi Chemical Corp.), a synthetic adsorbent of SEPABEADS SP series (e.g., SP207, SP207SS, SP20SS, SP70, SP700, SP825, SP850, etc.; all trade names, manufactured by Mitsubishi Chemical Corp.), and a synthetic adsorbent of Amberlite XAD series (e.g., XAD4, XAD2000, XADFPX66, XAD1180N, XAD2, etc.; all trade names, manufactured by Rohm and Haas Chemicals LLC). If the base resin structure of the resin adsorbent is acrylic, examples of such an adsorbent include a synthetic adsorbent such as HP2MGL (trade name, manufactured by Mitsubishi Chemical Corp.) or XAD7HP (trade name, manufactured by Rohm and Haas Chemicals LLC). Further, it is also possible to use a cation exchange resin in which the base resin structure of the resin adsorbent is styrene-based, such as, for example, an Na-form, K-form, or Ca-form of a resin of DIAION PK series (e.g., PK208, PK216, PK220, etc.; all trade names, manufactured by Mitsubishi Chemical Corp.) or a resin of DIAION UBK series (e.g., UBK10, UBK12, UBK16, etc.; all trade names, manufactured by Mitsubishi Chemical Corp.). Further, it is also possible to use an ODS resin (octadecylsilyl group-bonded silica gel resin) or the like in which the base resin structure of the resin adsorbent is silica gel-based, a Sephadex resin (e.g., Sephadex LH20, Sephadex G-25), and the like.

In the separation and purification resin treatment method, the aqueous solvent liquid extract of the monk fruit obtained in the extraction step is passed through the separation and purification resin such as a styrene-divinylbenzene-based synthetic adsorbent, and then the adsorbed mogroside V is eluted to obtain an adsorbent treated liquid. The eluent used in this process is 30% (v/v) to 80% (v/v), preferably 35% (v/v) to 60% (v/v), more preferably 40% (v/v) to 50% (v/v) ethanol water.

As a specific application example of the separation and purification resin treatment method, the aqueous solvent liquid extract is passed through the styrene-divinylbenzene-based synthetic adsorption resin at a rate of 83 mL/min, followed by washing with ion-exchanged water. Next, relatively highly polar components including proteins and polyphenols other than mogroside components are eluted by allowing 20% (v/v) ethanol water to pass through the resin at a rate of 83 mL/min, and then 15 L of 50% (v/v) ethanol water is passed through the resin at a rate of 83 mL/min to elute the mogroside components, thereby obtaining an eluent. Thereafter, the solvent of this eluent is appropriately removed using a rotary evaporator.

Next, an ion exchange resin treatment method allowed to be used for food is applied to the separation treated liquid obtained by application of the separation and purification resin treatment method. Applying the ion exchange resin treatment method after the separation and purification resin treatment method can more effectively remove, in particular, peptides, proteins, polyphenols, and the like, which have ionizable structures, and makes it possible to obtain a refined monk fruit crude liquid extract.

In the ion exchange resin treatment method using a weakly basic anion exchange resin or the like, for example, the separation treated liquid is passed through the weakly basic anion exchange resin, which is a resin having primary to tertiary amino groups as functional groups in a styrene-based/acrylic base material, to obtain a fraction of the monk fruit crude liquid extract containing mogroside V. Note that the weakly basic anion exchange resin is preferably washed with water in advance to remove raw material monomers of the adsorbent and impurities in the raw material monomers.

As a specific application example of the weakly basic anion exchange resin treatment method, the adsorbent treated liquid is treated with the weakly basic anion exchange resin. The adsorbent treated liquid is passed through the weakly basic anion exchange resin, which is activated in the Na-form, at a rate of 17 mL/min, and the resulting eluent is dried to obtain a monk fruit crude extract.

Note that, in the separation and purification resin treatment method or the ion exchange resin treatment method, the resin treatment can be performed using one or more types of resin adsorbents. One type of resin adsorbent may be used, or two or more types of resin adsorbents may be used.

Next, a simulated moving bed (SMB) method, a nanofiltration membrane method, or both thereof, are applied to the solution including the monk fruit crude extract obtained by application of the ion exchange resin treatment method to recover a fraction containing mogroside V at a high concentration. This fraction has the mogroside V content of 65% by mass to 85% by mass, the protein content of 1% by mass or less, and the polyphenol content of 0.1% by mass or less, and preferably has the total content of isomogroside V, mogroside IV, and siamenoside I of 7% by mass to 15% by mass. In this manner, the monk fruit extract of the present disclosure including mogroside V at a high concentration can be efficiently obtained.

The simulated moving bed method is a system in which mogroside V, which is the target component, is continuously separated using an SMB device in which a plurality of columns are connected in an annular form via an eight-way valve or the like. Mogroside V is continuously separated by continuously injecting the solution containing the raw material monk fruit extract, making it possible to obtain mogroside V at a high concentration with high productivity. Supply and taken-out ports, namely, a feed port for supplying a sample solution, an eluent port for supplying an eluent, an extract port for taking out a solution rich in strongly adsorbed components, and a raffinate port for taking out a solution rich in weakly adsorbed components, are usually connected to a circulation path of the SMB device, dividing the circulation path into four sections. The column is preferably filled with the styrene-divinylbenzene-based synthetic adsorbent. Further, the eluent is 30% (v/v) to 80% (v/v), preferably 35% (v/v) to 60% (v/v), more preferably 40% (v/v) to 50% (v/v) ethanol water. Then, a solution fluid is taken out from the raffinate taken-out port of the third column (third section), and the output intensity at a UV wavelength of 203 nm (for detection of mogrosides, proteins, and polyphenols) is continuously monitored. When the fractions thus obtained are used for measuring mogroside V and proteins, two peaks of the eluted proteins and mogroside V are shifted from each other. When the peak of mogroside V (later fraction in time), in particular, the liquid volume corresponding to 30% to 100%, preferably 50% to 100%, more preferably 70% to 100% of the second half of the peak fraction of mogroside V is taken out, the monk fruit extract of the present disclosure rich in the mogroside V content can be obtained.

As a specific application example of the simulated moving bed method, a glass column with a diameter of 50 mm and a length of 50 cm is filled with 700 mL of the styrene-divinylbenzene-based synthetic adsorption resin, and four of the same columns are connected in series and used. As the eluent, 40 v/v % hydrous ethanol is circulated (recycling time of 280 min) through the four columns connected in series at a flow rate of 10 mL/min using a metering pump. To detect the components, absorbance detectors are set at outlets of the first to fourth columns of the SMB device, and the output intensity at a UV wavelength of 203 nm (for detection of mogrosides, proteins, and polyphenols) is continuously monitored. When proteins and mogroside V detected with UV 203 nm are measured in fractions of the first column outlet first eluate, both proteins and mogroside V are present, and their two eluted peaks are shifted from each other.

At the third column outlet of the SMB device, a liquid volume corresponding to about 70% of the first half of the protein peak and a liquid volume corresponding to about 70% of the second half of the mogroside V peak are recovered to the outside of the SBM system. The protein fraction does not include mogroside V and is thus discarded, while the mogroside V fraction has significantly reduced protein content. During this process, the developing solvent in the volume equivalent to that of the recovered liquid was added simultaneously with the recovery to continue the circulation. When the mixture of a peak A and a peak B remaining in the column system returns to the first column, 50 g of the 20% by mass refined monk fruit liquid is newly added. This operation is repeated to recover the liquid corresponding to 70% of the second half of the mogroside V peak, and the recovered liquid is powdered to obtain the monk fruit extract of the present disclosure.

On the other hand, the nanofiltration membrane method combines a pore separation (size separation) effect and an electrostatic separation effect due to the charge on the membrane surface to exhibit characteristic permeation performance, which makes it possible to obtain the monk fruit extract in which the target component mogroside V is further concentrated and other components are removed by permeation. Specifically, a nanofiltration membrane having a pore size of the nanofiltration membrane of approximately 1 to 50 nm and a low salt rejection rate of approximately 70% or less (e.g., NF membrane model number; NP010, sodium sulfate rejection rate of 25% to 40%, manufactured by Daicen Membrane-Systems Ltd.) is preferably used.

Examples of a material for the membrane include cellulose acetate, polyvinylidene fluoride, polytetrafluoroethylene, polysulfone, polyethersulfone, aromatic polyamide, hydrophilized polyamide, and a composite thereof. Aromatic polyamide, hydrophilized polyamide, and a composite thereof are preferable.

The shape of the membrane is not particularly limited, and examples of the shape include a spiral type, a hollow fiber type, a circular tube type, and a flat plate type. Of these, the spiral type, which can maintain a large membrane area, and the hollow fiber type, which has high separation accuracy, are considered suitable.

The thickness of the membrane is not particularly limited. However, from the viewpoint of water permeability, durability, and the like, the thickness is preferably 1 to 500 nm, more preferably 1 to 10 nm.

As a specific application example of the nanofiltration membrane method, 8 L of a 5% by mass aqueous solution of the monk fruit extract obtained by powdering the liquid corresponding to 70% of the second half of the mogroside V peak is used as a sample and applied to a nanofiltration (NF) membrane (sodium sulfate rejection rate of 25% to 40%, pore size: approximately 50 nm) and a nanofiltration (NF) membrane (sodium sulfate rejection rate of 85% to 95%, pore size: approximately 2 nm). The sample was added to a reaction tank (NP010 membrane reaction tank) in which the NP010 membrane is placed to perform membrane filtration under the conditions of a circulation pressure of 0.3 MPa, a circulation flow rate of 5 L/min, and 35° C., and the permeate is transferred to a reaction tank (NP030 membrane reaction tank) in which the NP030 membrane is placed. The transferred NP010 membrane permeate is subjected to a membrane treatment under the conditions of a circulation pressure of 0.5 MPa, a circulation flow rate of 5 L/min, and 35° C., and the NP030 membrane permeate is returned to the NP010 membrane reaction tank to perform a recycling operation for 5 hours. In this process, the circulation pressure is finely adjusted so that the amount of the permeates of both membranes is the same. Through this operation, proteins and polyphenols with large molecular weight are separated on the NP010 membrane reaction tank side, and low molecular weight components including mogroside V are separated on the permeate side (same as the NP030 membrane reaction tank side). Further, lower molecular components (amino acids, salts, etc.) than mogrosides permeate to the permeation side of the NP030 membrane (same as the NP010 membrane reaction tank side). Through this operation, the highly refined liquid of the monk fruit is concentrated in the NP030 membrane reaction tank. The obtained NP030 membrane reaction tank liquid is microfiltered (0.45 μm) and powdered using a powder dryer (spray dryer, conditions: inlet temperature of 180° C., outlet temperature of 80° C.) to obtain the monk fruit extract of the present disclosure.

### EXAMPLES

Hereinafter, the present disclosure will be described in detail using Examples. Needless to say, the present disclosure is not limited to the following Examples.

### Example 1

### (Preparation of Monk Fruit Extract)

Five kilograms of dried monk fruit raw materials and 100 L of ion-exchanged water were placed in a stainless-steel container (150 L), heated at 95° C. for 4 hours with stirring, and cooled to 25° C. The residue was removed using a wire mesh colander (30 mesh), and the remaining was filtered using a filter paper (pore size 1 μm) to obtain 90 L of a liquid extract. This liquid extract was treated with a styrene-divinylbenzene-based synthetic adsorption resin (manufactured by Mitsubishi Chemical Corp., Diaion HP20 (trade name), 5000 mL). Ninety liters of the liquid extract was passed through the activated Diaion HP20 (after 7500 mL of 4% NaOH water was passed through the resin at a rate of 83 mL/min, the resin was washed with 15 L of ion-exchanged water. Next, after 7500 mL of 5% H₂SO₄ water was passed through the resin at a rate of 83 mL/min, the resin was washed with 15 L of ion-exchanged water) at a rate of 83 mL/min, and the resin was washed with 15 L of ion-exchanged water.

Next, relatively highly polar components including proteins and polyphenols other than mogroside components were eluted by allowing 15 L of 20% (v/v) ethanol water to pass through the resin at a rate of 83 mL/min, and then 15 L of 40% (v/v) ethanol water was allowed to pass through the resin at a rate of 83 mL/min to elute the mogroside components, thereby obtaining 15 L of an eluent.

Further, ethanol in the eluent was removed by vacuum concentration (rotary evaporator) to obtain 5 L of a monk fruit crude liquid extract. The contents of a monk fruit extract obtained by powdering this monk fruit liquid extract were as follows. Mogroside V content: 52.2% by mass, isomogroside V content: 2.6% by mass, mogroside IV content: 2.7% by mass, siamenoside I content: 1.4% by mass, protein content: 15.1% by mass, polyphenol content: 0.2% by mass (sample 1). This monk fruit crude liquid extract was treated with a weakly basic anion exchange resin (manufactured by Mitsubishi Chemical Corp., Diaion WA30 (trade name), 1000 mL). Five liters of the monk fruit crude liquid extract was passed through the activated Diaion WA-30 (after 1500 mL of 4% NaOH water was passed through the resin at a rate of 17 mL/min, the resin was washed with 3 L of ion-exchanged water) to obtain a monk fruit liquid extract. The contents of a monk fruit extract obtained by powdering this monk fruit liquid extract were as follows. Mogroside V content: 59.2% by mass, isomogroside V content: 3.0% by mass, mogroside IV content: 2.5% by mass, siamenoside I content: 1.2% by mass, protein content: 5.5% by mass, polyphenol content: 0.1% by mass (sample 2).

Next, the monk fruit powdered extract described above was dissolved in purified water to a concentration of 20% by mass. A refined monk fruit liquid thus obtained was purified with a simulated moving bed method.

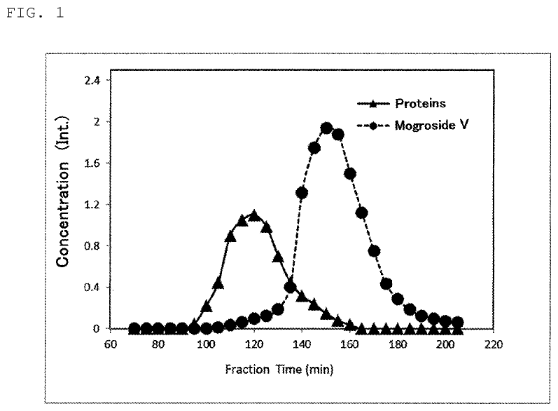

The SMB treatment conditions are as follows. Specifically, a glass column with a diameter of 50 mm and a length of 50 cm was filled with 700 mL of the styrene-divinylbenzene-based synthetic adsorption resin (manufactured by Mitsubishi Chemical Corp., Diaion HP20 (trade name)), and four of the same columns are connected in series and used. As the eluent, 40 v/v& hydrous ethanol was circulated (recycling time of 280 min) through the four columns connected in series at a flow rate of 10 mL/min using a metering pump. To detect the components, absorbance detectors were set at the outlets of the first to fourth columns of the SMB device, and the output intensity at a UV wavelength of 203 nm (for detection of mogrosides, proteins, and polyphenols) was continuously monitored. The measurement result of the protein content and mogroside V content of each fraction of the first column outlet first eluate was shown in FIG. 1.

As shown in FIG. 1, two eluted peaks (of proteins and mogroside V; shown in FIG. 1) were shifted but not completely separated from each other. In other words, the second half of the protein peak and the first half of the mogroside V peak overlapped. Thus, approximately 70% of the first half of the protein peak that did not include mogroside V was recovered and discarded at the third column outlet of the SMB device, and a 70% liquid volume corresponding to 30% to 100% of the mogroside V peak was recovered to the outside of the SBM system. Note that, in this process, the developing solvent in the volume equivalent to that of the recovered liquid was added simultaneously with the recovery to continue the circulation. This operation was repeated to recover a liquid corresponding to 70% of the second half of the mogroside V peak. The recovered liquid was powdered to obtain a highly refined monk fruit extract. The contents of this highly refined monk fruit extract were as follows. Mogroside V content: 65.8% by mass, isomogroside V content: 3.8% by mass, mogroside IV content: 3.2% by mass, siamenoside I content: 2.4% by mass, protein content: 0.8% by mass, polyphenol content: 0.08% by mass (Sample 3). As a result, mogroside V was highly purified.

Next, the mogroside V peak was confirmed with a monitor at the fourth column outlet of the SMB device, and at the same time as the peak returned to the first column of the SMB device, 50 g of the 20% by mass refined monk fruit liquid was newly added to continue the circulation, thereby repeating this operation. The newly charged monk fruit extract and 30% of the first half of the mogroside V peak were mixed and purified again by the SBM method without losing any mogrosides.

To measure the contents of mogroside V and mogrosides, approximately 0.05 g of the monk fruit sample was accurately weighed using a chemical balance, and the sample was added with distilled water up to 100 mL using a volumetric flask to obtain a solution used as an HPLC analysis sample. In the HPLC analysis [analysis conditions: column; SHODEX Asahipak NH2P-50 4E (4.6 mm ID×250 mm), developing solvent; 75% by mass CH₃CN/25% by mass H₂O, isocratic mode, flow rate; 0.8 mL/min, detection wavelength; 203 nm, charge volume; 10 μL], calibration curves at a concentration of 100 ppm to 600 ppm were created for a mogroside V standard (manufactured by Wako Pure Chemical Industries, Ltd.) and a mogroside standard (manufactured by Cosmo Bio) to measure the content of mogroside V in the sample. The protein content was measured by the Kjeldahl method. The polyphenol content was measured by the Folin-Ciocalteu method.

### Example 2

Using 8 L of a 5% by mass aqueous solution of the highly refined monk fruit powdered extract described in Example 1 as a sample, purification was performed with a nanofiltration (NF) membrane purification method. The purification was performed using a nanofiltration (NF) membrane (manufactured by Daicen Membrane-Systems Ltd., NF membrane model number; NP010, sodium sulfate rejection rate of 25% to 40%, pore size: approximately 50 nm) and a nanofiltration (NF) membrane (manufactured by Daicen Membrane-Systems Ltd., NF membrane model number; NP030, sodium sulfate rejection rate of 85% to 95%, pore size: approximately 2 nm). The sample was added to a reaction tank (NP010 membrane reaction tank) in which the NP010 membrane was placed to perform membrane filtration under the conditions of a circulation pressure of 0.3 MPa, a circulation flow rate of 5 L/min, and 35° C., and the permeate was transferred to a reaction tank (NP030 membrane reaction tank) in which the NP030 membrane was placed. The transferred NP010 membrane permeate was subjected to a membrane treatment under the conditions of a circulation pressure of 0.5 MPa, a circulation flow rate of 5 L/min, and 35° C., and the NP030 membrane permeate was returned to the NP010 membrane reaction tank to perform a recycling operation for 5 hours. In this process, the circulation pressure was finely adjusted so that the amount of the permeates of both membranes is the same. Through this operation, proteins and polyphenols with large molecular weight were separated on the NP010 membrane reaction tank side, and low molecular weight components including mogroside V were separated on the permeate side (same as the NP030 membrane reaction tank side). Further, lower molecular components (amino acids, salts, etc.) than mogrosides permeated to the permeation side of the NP030 membrane (same as the NP010 membrane reaction tank side). Through this operation, the highly refined liquid of the monk fruit was concentrated in the NP030 membrane reaction tank. The obtained NP030 membrane reaction tank liquid was microfiltered (0.45 μm) and powdered using a powder dryer (spray dryer, conditions: inlet temperature of 180° C., outlet temperature of 80° C.) to obtain a highly refined monk fruit extract. The contents of this highly refined monk fruit extract were as follows. Mogroside V content: 73.2% by mass, isomogroside V content: 4.3% by mass, mogroside IV content: 4.2% by mass, siamenoside I content: 1.4% by mass, protein content: 0% by mass, polyphenol content: 0% by mass (Sample 4).

Further, a 50% liquid volume (sample 5) corresponding to 50% to 100% of the mogroside V peak shown in the drawing of Example 1, a 30% liquid volume (sample 6) corresponding to 50% to 80%, and a 10% liquid volume (sample 7) corresponding to 50% to 60% were treated with the method in Example 2, and then these samples were powdered and analyzed for each content. The contents of the sample 5 were as follows. Mogroside V content: 84.9% by mass, isomogroside V content: 4.2% by mass, mogroside IV content: 3.5% by mass, siamenoside I content: 1.1% by mass, protein content: 0% by mass, polyphenol content: 0% by mass. The contents of the sample 6 were as follows. Mogroside V content: 90.1% by mass, isomogroside V content: 2.1% by mass, mogroside IV content: 1.2% by mass, siamenoside I content: 1.3% by mass, protein content: 0% by mass, polyphenol content: 0% by mass. The contents of the sample 7 were as follows. Mogroside V content: 95.8% by mass, isomogroside V content: 0.2% by mass, mogroside IV content: 0% by mass, siamenoside I content: 0% by mass, protein content: 0% by mass, polyphenol content: 0% by mass.

### Example 3

### (Evaluation of Sweetness Intensity and Sweetness Quality)

The powdered monk fruit extract prepared in Example 1 (sample 1: mogroside V content of 52.2% by mass) and the highly refined monk fruit extracts prepared in Example 1 (sample 2: mogroside V content of 59.2% by mass, sample 3: mogroside V content of 65.8% by mass) and Example 2 (sample 4: mogroside V content of 73.2% by mass, sample 5: mogroside V content of 84.9% by mass, sample 6: mogroside V content of 90.1% by mass, sample 7: mogroside V content of 95.8% by mass) were examined for sweetness intensity, bitterness quality, and sweetness quality.

The sweetness evaluation test was performed by diluting each sample with distilled water so that the mogroside V concentration is adjusted to 228 ppm. In this manner, a total of 7 sample solutions were prepared. An aqueous solution containing sucrose at a concentration of 10% was used as a control. The sweetness intensity was evaluated on a 10-point scale with sucrose being 10, the bitterness intensity was evaluated on a 10-point scale with sucrose being 5, and the sweetness quality was evaluated on a 10-point scale for the basic sweetness features including the following items: mellowness, aftertaste, bitterness, off-flavor, richness, astringency, and pungency. A sensory test was performed by 10 panelists, and each item was shown as an average score in comparison to the sucrose aqueous solution. The results are shown in Table 1.

TABLE 1

| | Sweetness | Bitterness | Sweetness | |
|---|---|---|---|---|
| Samples | intensity | intensity | evaluation | Evaluation |
| Sucrose | 1.0 | 5.0 | 10 | Control |
| (10%) | | | | |
| Sample 1 | 6.2 | 8.2 | 6.7 | Less sweet, |
| | | | | bitter |
| | | | | aftertaste. |
| Sample 2 | 7.1 | 7.5 | 7.3 | Less sweet, |
| | | | | short- |
| | | | | lasting |
| | | | | (followed |
| | | | | by off- |
| | | | | flavor) |
| Sample 3 | 7.3 | 6.7 | 8.1 | Sweetness |
| | | | | similar to |
| | | | | sucrose |
| | | | | (some off- |
| | | | | flavor) |
| Sample 4 | 9.0 | 6.2 | 9.2 | Sweetness |
| | | | | similar to |
| | | | | sucrose |
| | | | | (long- |
| | | | | lasting |
| | | | | sweetness |
| | | | | with little |
| | | | | off-flavor) |
| Sample 5 | 9.0 | 6.2 | 9.1 | Sweetness |
| | | | | similar to |
| | | | | sucrose |
| | | | | (long- |
| | | | | lasting |
| | | | | sweetness |
| | | | | with little |
| | | | | off-flavor) |
| Sample 6 | 7.3 | 6.3 | 7.5 | Low |
| | | | | sweetness |
| | | | | intensity |
| Sample 7 | 7.1 | 6.0 | 7.6 | Low |
| | | | | sweetness |
| | | | | intensity |

As is clear from the results shown in Table 1, the sample 3, the sample 4, and the sample 5 were superior to the sample 1 and the sample 2 in the sweetness intensity, the bitterness evaluation, and the sweetness quality evaluation. Further, the sample 6 and the sample 7, which had the lower total content of isomogroside V, mogroside IV, and siamenoside I, were inferior to the sample 3, the sample 4, and the sample 5 in the sweetness intensity, the bitterness evaluation, and the sweetness quality evaluation. As a result, it was found that the monk fruit extract as a product of the present disclosure exhibited the high sweetness quality, had little off-flavor as aftertaste, and had no bitterness.

### Example 4

### (Evaluation of Clarity of Aqueous Solution)

After adding 9 g of distilled water to 1 g of each of the samples 1 to 7 described in Example 3 and mixing the mixture uniformly, the absorbance at 660 nm was measured using a spectrophotometer with an optical path length of 1 cm (1 cm cell). Distilled water was used as a control. Measurements were performed in triplicate for each sample, thereby determining the average value. As a result, the average absorbance values of the samples 1 to 7 were 0.838, 0.153, 0.032, 0.007, 0.003, 0.002, and 0.002, respectively. The sample 3 to the sample 7 had lower absorbance, had less insoluble matters, and had higher clarity than the sample 1 and the sample 2.

1. A monk fruit (Siraitia grosvenorii) extract comprising:
(a) 65% by mass to 85% by mass of mogroside V in the extract;
(b) 1% by mass or less of a protein in the extract; and
(c) 0.1% by mass or less of a polyphenol in the extract.

2. The monk fruit extract according to claim 1, comprising:
(a) 71% by mass to 85% by mass of the mogroside V in the extract;
(b) 0.5% by mass or less of the protein in the extract; and
(c) 0.08% by mass or less of the polyphenol in the extract.

3. The monk fruit extract according to claim 1, wherein the monk fruit extract has a total content of isomogroside V, mogroside IV, and siamenoside I of 7% by mass to 15% by mass.

4. The monk fruit extract according to claim 1, wherein the monk fruit extract includes 71% by mass to 85% by mass of the mogroside V, 1% by mass or less of the protein, and 0.1% by mass or less of the polyphenol, and has the total content of the isomogroside V, the mogroside IV, and the siamenoside I of 7% by mass to 15% by mass.

5. The monk fruit extract according to claim 1, wherein an absorbance at a wavelength of 660 nm in a 10% by mass aqueous solution is 0.1 or less.

6. A sweetener composition comprising the monk fruit extract according to claim 1, as an active ingredient.

7. An antioxidant comprising the monk fruit extract according to claim 1, as an active ingredient.

8. A food or drink product comprising the monk fruit extract according to claim 1, as an active ingredient.

9. A quasi-drug comprising the monk fruit extract according to claim 1, as an active ingredient.

10. A cosmetic comprising the monk fruit extract according to claim 1, as an active ingredient.

11. A method for producing the monk fruit extract according claim 1, comprising;
a step of extracting monk fruit with an aqueous solvent;
a step of applying a styrene-divinylbenzene-based synthetic adsorbent treatment method to an aqueous solvent liquid extract of the monk fruit obtained in the extraction step;
a step of applying a weakly basic anion exchange resin treatment method to an adsorbent treated liquid obtained by application of the styrene-divinylbenzene-based synthetic adsorbent treatment method; and
a step of applying a simulated moving bed method or a nanofiltration membrane method, or both thereof, to a solution including a monk fruit crude extract obtained by application of the weakly basic anion exchange resin treatment method, thereby adjusting a content of mogroside V to 65% by mass to 85% by mass.

12. A method for producing a monk fruit extract, comprising:
a step of extracting monk fruit with an aqueous solvent; and a step of applying one or more types of separation and purification resin treatment methods allowed to be used for food to an aqueous solvent liquid extract of the monk fruit obtained in the extraction step, wherein the monk fruit extract includes:
(a) 65% by mass to 85% by mass of mogroside V in the extract
(b) 1% by mass or less of a protein in the extract; and
(c) 0.1% by mass or less of a polyphenol in the extract.

13. The method for producing the monk fruit extract according to claim 12, comprising:
a step of extracting the monk fruit with the aqueous solvent; and
a step of applying a nanofiltration membrane method to a solution including a monk fruit crude extract obtained by applying the one or more types of separation and purification resin treatment methods allowed to be used for food to the aqueous solvent liquid extract of the monk fruit obtained in the extraction step.

14. The method for producing the monk fruit extract according to claim 12, wherein the one or more types of separation and purification resin treatment methods include a simulated moving bed method.

15. The method for producing the monk fruit extract according to claim 12, wherein a resin adsorbent used in the separation and purification resin treatment methods is at least one selected from the group consisting of a synthetic adsorbent, an ion exchange resin, an ODS resin, and a Sephadex resin.
